(19) United States
(12) Patent Application Publication
(10) Pub. No.: US 2026/0327539 A1
(43) Pub. Date: Oct. 22, 2026

(54) APPARATUS AND METHODS FOR RESEARCH OF A CELLULAR COMMUNITY

(71) Applicants: Mehdi Mohammadi ASHANI, Calgary (CA); Stephanie Lauren BISHOP, London (CA); Ian Andrew LEWIS, Calgary (CA)

(72) Inventors: Mehdi Mohammadi ASHANI, Calgary (CA); Stephanie Lauren BISHOP, London (CA); Ian Andrew LEWIS, Calgary (CA)

(21) Appl. No.: 19/115,006

(22) PCT Filed: Jun. 5, 2023
(86) PCT No.: PCT/CA2023/050767

§ 371 Date: Mar. 25, 2025

Related U.S. Application Data
(60) Provisional application No. 63/412,121, filed on Sep. 30, 2022.

(51) Int. Cl. C12M 1/32; C12M 1/12; C12M 1/34

(52) U.S. Cl. CPC C12M 23/12; C12M 25/04; C12M 41/36

(57) ABSTRACT

A laboratory apparatus for research of a microbial community comprising: a receiver plate with a well; an insert plate for securing over the receiver plate, the insert plate including a plurality of tubes extending from the lower surface, each of the plurality of tubes including an upper open end and a lower end that is open, the plurality of tubes are configured such that the lower ends protrude into the well; and a membrane extending across the lower end of each of the plurality of tubes, the membrane selected to exclude a cultured biological moiety, while permitting molecules smaller than the cultured biological moiety of a culture medium to pass through. Methods for culturing biological moieties are also described.

### FIELD

This invention relates to a laboratory apparatus and methods for organism culturing, and studying the contribution of individual cell types in a community

### BACKGROUND

Interactions between biological moieties such as microorganisms (microbes), cells, viruses, etc. in a community, including the exchange of nutrients, play a vital role in human health and have a great impact on ecological niches. However, due to the complexity of the metabolic interactions in microbial communities, it is difficult to study the individual metabolic contributions of each microbe to a community. There are few if any devices available to facilitate such studies. This has hindered the ability to assess these important polymicrobial interactions that are prevalent in many environmental niches. Technical innovations that address this shortcoming would accelerate the pace of research on polymicrobial interactions.

### SUMMARY

A laboratory apparatus and methods for research of biological moiety communities have been invented. The biological moieties are viruses or cells including cell lines, mammalian cells, plant cells, immune cells, organisms, microorganisms including prokaryotic cells, bacteria or yeasts, etc.

The invention permits the study of a community, for example their interactions and, for example, is for the direct study of individual metabolic contributions of biological moieties to a community metabolite pool. The invention permits the measurement of both metabolites/signals from one kind of biological moiety and the metabolites of the biological moiety community.

In accordance with a broad aspect of the present invention, there is provided a laboratory apparatus for research of a biological community comprising:
a. a receiver plate with a receiver well;
b. an insert plate for positioning over the receiver plate, the insert plate including a first tubular well extending from the lower surface and a second tubular wall extending from the lower surface, each of the first and second tubular wells including an upper open end and a lower end that is open, the first and second tubular wells are configured such that the lower ends protrude into the receiver well when the insert plate is positioned over the receiver plate; and
c. a first membrane extending across the lower end of the first tubular well and a second membrane extending across the lower end of the second tubular well, the first membrane selected to exclude a first cultured biological moiety, while permitting molecules smaller than the first cultured biological moiety of a culture medium to pass through and the second membrane selected to exclude a second cultured biological moiety, while permitting molecules smaller than the second cultured biological moiety of a culture medium to pass through.

In accordance with another broad aspect of the present invention, there is provided a method for researching biological systems with a laboratory apparatus as described above, comprising one or more of:
seeding a first cell type into the first tubular well and seeding a biological moiety into at least one of the receiver well and the second tubular well; and determining a metabolic contribution of the first cell type and the biological moiety to an ensemble phenotype, wherein a concentration of a metabolite is measured independently in both the first tubular well and the at least one of the receiver well and the second tubular well; and
seeding a first cell type into the first tubular well and seeding a biological moiety into at least one of the receiver well and the second tubular well; and assessing growth of the first cell type.

In accordance with another broad aspect of the present invention, there is provided a method for researching biological communities in culture, the method comprised of:
a. adding culture medium to a container system including a first well, a second well and the a third well, wherein the first well and the second well are in fluid communication through a first semipermeable membrane and wherein the first well and the third well are in fluid communication through a second semipermeable membrane, and adding culture medium to the container system adds culture medium to at least make contact with the first and the second semipermeable membranes and wherein the culture medium contains a molecule that can pass through the first and the second semipermeable membranes; and
b. seeding a first biological moiety into the second well, the first biological moiety having a size that is unable to pass through the first semipermeable membrane;
c. seeding a second biological moiety into the third well, the second biological moiety having a size that is unable to pass through the second semipermeable membrane; and
d. sampling culture medium from the second well.

It is to be understood that other aspects of the present invention will become readily apparent to those skilled in the art from the following detailed description, wherein various embodiments of the invention are shown and described by way of example. As will be realized, the invention is capable for other and different embodiments and several details of its design and implementation are capable of modification in various other respects, all captured by the present claims. Accordingly, the detailed description and examples are to be regarded as illustrative in nature and not as restrictive.

### DESCRIPTION OF THE FIGURES

For a better appreciation of the invention, the following Figures are appended:

FIG. 1A is an exploded perspective view of an apparatus for culturing organisms;

FIG. 1B is a side elevation view of the assembled apparatus of FIG. 1A;

FIG. 2A is an exploded perspective view of another apparatus for culturing organisms;

FIG. 2B is a perspective view of the assembled apparatus of FIG. 2A;

FIG. 3 is are perspective views of a plurality of different receiver plate configurations;

FIG. 4A is a schematic view of an example method in an apparatus for culturing organisms. For reference, the overall compartment structure shown in FIG. 4A is similar to the compartment within rectangle 4a of FIG. 1B;

FIG. 4B is a diagram showing a calculation for a change in molecule concentration between an individual well and a community well;

FIG. 4C is a diagram showing the calculation for the net contribution of a biology moiety to the collective molecule pool, wherein there are multiple biological moieties present and multiple molecules exchanged in the community well;

FIG. 5A is a schematic with an enlarged inset of another apparatus for culturing organisms;

FIG. 5B are microscope images from Example 3, of MRC-5 cell line cells incubated for 72 hrs without infection (left hand side) and infected with Human Coronovirus after 72 hours (right hand side).

FIG. 5C is a PCR sterility test result, as in Example 3, for Human Coronavirus: in the receiver well infected with virus (Bottom+HRV), receiver well without viral infection (Bottom−HRV), insert well with cell and coronavirus in the receiver well (Top+HRV) and insert well with only cell and no viral infection in the receiver well (Top−HRV).

FIGS. 6A and 6B relate to a diffusion simulation in Example I, where FIG. 6A is a 3D model with geometry and location of the cross-sectional sheet and the sampling zone; and FIG. 6B is a diffusion graph over AB line for 0-9 hours showing molar concentration over the cross-sectional sheet.

FIG. 7 shows an example of an experimental setup wherein the apparatus is designed to house six individual wells in the insert plate connected through a semi-permeable to one community well in the receiver plate. In this example, the control sample was the growth medium (BHI medium), which contained no biological moieties. The microbial species used in this setup included Haemophilus influenzae, Pseudomonas aeruginosa, and Staphylococcus aureus. In all compartments other than the control compartment containing only growth medium, either one, two, or three species of microorganisms were housed in compartments connected through a community well in the receiver plate that contained only BHI growth medium and no microbial species. Compartments that contained only one microbial species in the six individual wells of the insert plate and, thus, only exchanged molecules among one species in the receiver well were considered monocultures. Compartments were identified as community cultures where in the top wells, at least one well had one species and at least a second well had a different species, and where that first and second top well were both in communication with the same receiver well. There were community cultures with two and three microbial species in the six individual wells of the insert plate, and which exchanged molecules between multiple species in the receiver well.

FIG. 8 contains plots showing microbial growth in the apparatus, as described in Example 2, over a 24-hour period. Colony forming units (CFU)/mL counts at each time point were taken by sampling a 20 μL aliquot from individual wells housing microbial species and diluting each aliquot by a factor of 10²-10⁶, followed by plating the diluted cells. Aliquots were taken from compartments containing one microbial species in the insert plate (monoculture) and from compartments containing three insert wells each had different microbial species in them but shared the same receiver plate (community culture). H—Haemophilus influenzae; P—Pseudomonas aeruginosa; S—Staphylococcus aureus.

FIGS. 9A and 9B are a heatmap showing initial screening of metabolites at the 9-hour time point, demonstrating patterns of metabolite consumption and production by different microbial species in a monoculture (one microbial species present in the community) and community cultures (three microbial species present in the community). The growth medium control (BHI) values are shown on the left. Metabolite values are normalized by z-score of the metabolite concentrations across each row with values shown in the legend. H—Haemophilus influenzae; P—Pseudomonas aeruginosa; S—Staphylococcus aureus.

FIG. 10 is a series of bar plots showing metabolite concentrations at each time point, with error bars showing standard error of the mean. For each treatment, n=6-9 replicates and statistical differences between treatments were calculated by one-way ANOVA with post-hoc Tukey correction, where *p<0.05; **p<0.01; ***p<0.001; and ****p<0.0001 (only differences between the same microbial species in the monoculture samples compared to the community culture samples or concentrations in individual wells in the insert plate compared to the community well in the receiver plate (HPS) are shown). Sample legend: medium control—BHI; top well (insert plate) monocultures—H (Haemophilus influenzae), P (Pseudomonas aeruginosa), S (Staphylococcus aureus); bottom well (receiver plate) three-species culture—HPS; and top well (insert plate) three-species community cultures H* (H-HPS), P* (P-HPS), S* (S-HPS).

FIG. 11 shows the calculated metabolic flux, defined as the change in metabolite concentration over time, between three microbial species in a compartment containing three species housed in individual wells of the insert plate (H. influenzae, P. aeruginosa, and S. aureus) and a community well on the bottom (receiver) plate where the exchange of metabolites occurs. Flux values are normalized to the relative concentration of each metabolite and represented by the arrows, indicating both direction and magnitude of flux. This shows the overall metabolic contribution of individual microbial species to the community and visualizes the flow of metabolites from one microbial species to another microbial species in the community.

FIGS. 12A and 12B are illustrations of the biological interpretation of data generated in Example 2 and shown in detail in FIG. 11, wherein FIG. 12A shows normalized metabolite flux values for the three microbial species in the community, wherein the arrow thickness denotes value of metabolite flux and FIG. 12B is a metabolic pathway mapping of certain metabolites exchanged in the example community ecosystem. Abbreviations: Thr-threonine, Cit-citrulline, Asp-aspartate, G3P-glycerol 3-phosphate, Arg-arginine, DHAP-dihydroxyacetone phosphate.

FIG. 13 are diagrams from Example 3, which is a metabolomics study for drug testing (Remdesivir), MRC-5 cell and Coronavirus 229 in the apparatus. The diagrams are for (a) Hypoxanthine, (b) Inosine, (c) Uridine and (d) Alpha-D-Glucose.

FIG. 14 is a chart showing the biofilm formation from two and three bacteria interactions, as described in Example 4.

FIG. 15 are microscope images of Caco2 and HT29-MTX-E12 cells grown in receiver wells after 24 hours.

FIGS. 16A to 16C are schematic views of example culturing set ups.

### DETAILED DESCRIPTION OF VARIOUS EMBODIMENTS

The detailed description and examples set forth below are intended as a description of various embodiments of the present invention and are not intended to represent the only embodiments contemplated by the inventor. The detailed description includes specific details for the purpose of providing a comprehensive understanding of the present invention. However, it will be apparent to those skilled in the art that the present invention may be practiced without these specific details.

The apparatus and methods enable the exchange of nutrients between a plurality of different, but individually-housed biological moieties, such as microbes, cell cultures and viruses, in a shared community metabolite pool.

The apparatus includes a top plate and a bottom plate. The top plate fits onto the bottom plate. The top plate includes a plurality of spaced apart wells. Each well has an open top and a lower end and is sized to accommodate a culture containing a biological moiety. While the lower end is open, a semi-permeable membrane is installed on the lower end. Thus, the lower end is open to passage of molecules that can pass through the membrane.

The bottom plate includes a plurality of wells, where each well is defined by walls and each has an open top. More than one upper well can protrude into each lower plate well. In use, culture media is added to the wells such that the semi-permeable membranes are submerged in the media when the top plate is fit onto the lower plate. Biological moieties are seeded into the top wells and/or the bottom well and cultured therein. Cellular communities cultured in the individual microbe wells on the top plate are physically separated from each other by the semi-permeable membranes. However, the cellular communities are in communication at a molecular level, through a shared culture, metabolite pool provided in the shared well of the bottom plate. Metabolites can freely diffuse through the semi-permeable membranes, allowing the exchange of molecules between different members of the community from top well to top well. Metabolites can be extracted both from the individual and community wells separately, allowing researchers to investigate the effects of poly-organism metabolism on individual organism metabolism. The flexibility and simple design of the apparatus enables a multitude of experimental configurations and model systems for research which were not previously possible.

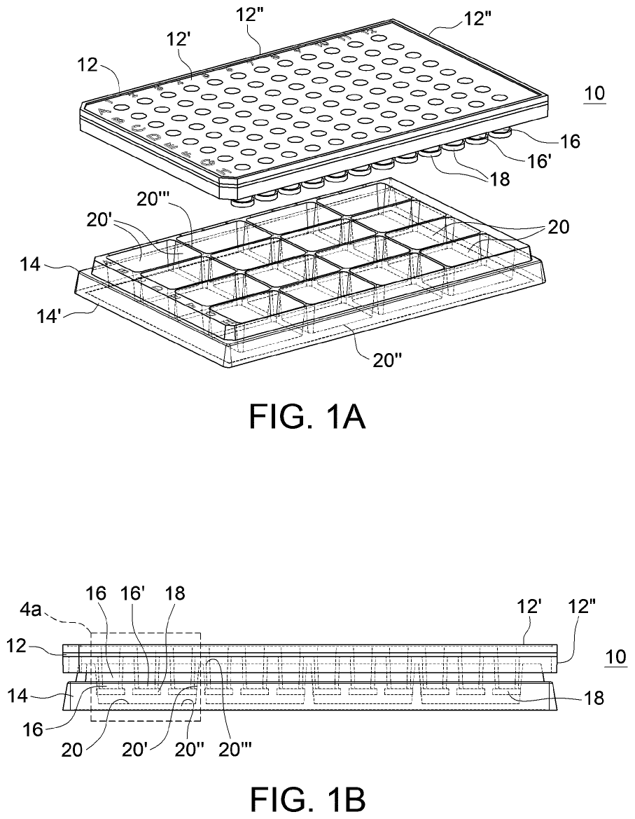

With reference to FIGS. 1A and 1B, the apparatus 10 includes a top plate, also called an insert plate 12, and a bottom plate, also called a receiver plate 14.

The insert plate 12 has protruding from its underside a plurality of tubular wells 16, each tubular well is spanned at its bottom end 16′ by a membrane 18. The insert plate 12 securely fits over the receiver plate 14. The receiver plate 14 contains at least one well 20 sized to receive at least two of the insert plate's tubular wells 16. In particular, when the insert plate 12 is fit onto the receiver plate, the bottom ends 16′ and membranes 18 of at least two of the tubular wells 16 protrude down into one of the wells 20 on the receiver plate. The at least two tubular wells 16 protrude down into the same well 20.

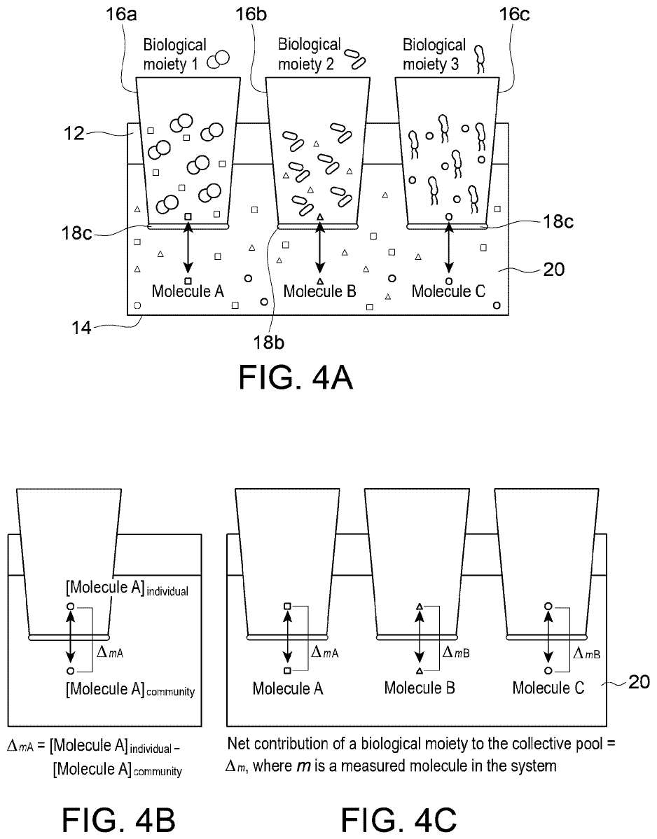

As such, apparatus 10 permits controlled molecular diffusion, and thereby communication, between the contents of well 20 and the contents of each of the individual tubular wells 16, as limited by the filtering action of the membrane. The diffusion is of molecules with an effective size or condition able to pass through membrane 20. The molecules can be liquids, gases, proteins, carbohydrates, lipids or inorganics such as salts, which may include signalling molecules, metabolites and metabolic waste products. In one embodiment shown in FIG. 4, where there are a first tubular well 16a, a second tubular well 16b and a third tubular well 16c protruding into the same receiver well 20, there may be controlled diffusion from the contents of each of the three tubular wells 16a, 16b, 16c to the contents of the receiver well 20 and, therethrough, controlled diffusion from the first tubular well 16a to the second and third tubular wells 16b, 16c of the insert plate, all controlled diffusion of which is limited by the action of the membranes 18a, 18b, 18b. The membranes may each be the same or different.

One configuration of the device includes an insert plate with 2 to 400 tubular wells 16 on a plate format. The corresponding receiver plate has one or more wells 20 dimensioned groups to receive all or smaller groups of two or more of the insert plate's tubular wells 16. Further, the receiver plate can have enough wells spread out on its surface to receive all of the tubular wells on the insert plate in these small groups of two to 12. The tubular wells in each group are connected to each other through their membranes 18 and through the common receiver plate well in which the group of tubular wells reside.

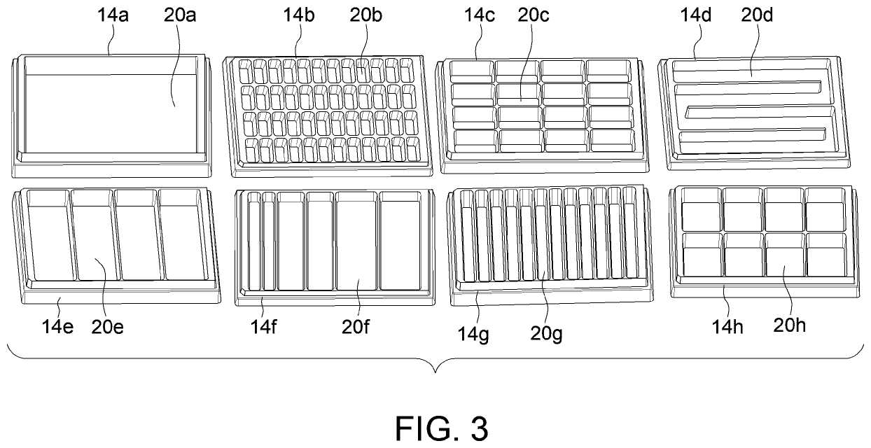

In the illustrated embodiment, of FIG. 1A there are 96 tubular wells 16 on the insert plate 12 and sixteen wells 20 on the receive plate 14. The arrangement of tubular wells 16 and wells 20, configure the apparatus 10 to have six tubular wells 16 protruding into each receiver well 20. It is to be understood that many other configurations for this device are possible. For example, some insert plates have six, 24, 96 or 384 tubular wells. As shown in FIG. 3, various receiver plates 14a-14h with different well 20a-20h shapes and arrangements are possible. It may facilitate manufacture by constructing an insert plate that can fit over more than one different style of receiver plate, with various configurations of wells.

The receiver plate 14 has perimeter edges 14′ and includes the one or more wells within its perimeter edges. The well of the lower plate, as will be appreciated, has side walls 20′ extending between a bottom wall 20″ and an upper rim 20″′ with a depth being defined between the bottom wall and the upper rim and a volume being defined between the side walls. The well is often a rectangular/square shape but this can vary, as shown in FIG. 3.

The insert plate of the device includes an upper wall 12′ extending between side edges 12″. The upper wall includes an upper surface, a lower surface and the tubes 16 extend from the lower surface.

The insert plate 12 is configured to fit onto the receiver plate while the upper ends of the tubular wells 16 are maintained above the volume of the receiver wells. Therefore, edges 12″ of the insert plate may extend laterally beyond the well 20 upper rim 20″′ toward the perimeter edges 14′ of the receiver plate 14 and edges 12″ can be supported on perimeter edges 14′. This configuration may support the tubular wells with their lower ends 16′ supported spaced from a bottom wall 20″ of the well 20. For example, the lower end 16′ has an outer diameter smaller than the diameter of the well in which it is to reside. While there are many ways to shape the insert plate, the tube is shaped and configured with a length that positions the lower end within the depth of the receiver plate well but spaced from the end wall.

As noted, membrane 18 extends fully across the open, lower end 16′ of each tube of the insert plate. The membrane is secured as by sealing against the lower end of the tube. The membrane creates an impermeable barrier to passage therethrough of the biological moiety, but permits diffusion of molecules such as, for example, metabolites, proteins, signalling molecules and metabolic waste products. Thus, the cultured biological moiety is trapped in either the insert well or the receiver well and cannot move past the bottom end, and in particular the membrane, of the insert well. The cultured biological moiety can include, for example, a virus or a cell. The cell may be a cell line, plant cell, mammalian cell, immune cell, yeast, prokaryotic cell, etc. that are capable of being cultured in a culture medium.

The membrane, being semi-permeable, has an exclusion rating. The exclusion rating may include aperture size exclusions, chemical exclusions, etc., and is selected based on the known characteristics of the biological moieties to be cultured for example, the known size or surface chemistry of the microbe, but is large enough to permit passage of other culture components: molecules, liquids and gasses, as desired. Since the membrane is on the lower end of the tube and, when the insert plate and the receiver plate are assembled, the lower end protrudes into the receiver well, the membrane securely segregates the interior of the tube from the interior of the receiver well, according to the exclusion rating of the membrane. In particular, again with reference to FIG. 4A, the apparatus creates at least three compartments for each well: (i) a lower compartment, which is in the well 20; (ii) a first upper compartment within the first tube 16a separated from the lower compartment by its membrane 18a and (iii) a second upper compartment within the second tube 16b separated from the lower compartment by its membrane 18b. Each of the first and second compartments are open to the upper surface of the insert plate and, thereby, open to the exterior of the device. When there are microbial cultures in the apparatus, the membrane securely segregates cultured organisms in the individual tubular wells 16a, 16b of the insert plate from the culture medium wells in the receiver plate while allowing culture non-excluded molecules to pass through. The selected, non-excluded culture components, such as liquids, gases and small molecules such as proteins, carbohydrates, lipids or inorganics such as salts, which may include one or more signalling molecules and metabolites such as nutrients and by-products, can freely diffuse through the membrane between the upper and the lower compartments.

As shown in FIG. 4A, the membrane 18a across the lower end of tubular well 16a securely segregates cultured organism biological moiety 1 in well 16a and the membrane 18b across tubular well 16b securely segregates cultured organism biological moiety 2 in well 16b. These moieties 1 and 2 cannot move into the medium in well 20 due to the membranes 18a, 18b. However, non-excluded molecule A can diffuse, see arrow, through membrane 18a from tubular well 16a into the culture in well 20 and non-excluded molecule B can diffuse, see arrow, through membrane 18b from tubular well 16b into the culture in well 20. Thus, well 20 has a concentration of both molecules A and B. If desired, the membranes can also permit diffusion of molecules from well 20 into the tubular wells. As such, molecule B can diffuse into tubular well 16a. Thus, the effect of molecule B can be seen with respect to biological moiety 1.

FIG. 4A also shows a cultured organism biological moiety 3 in well 16c, which is excluded by and cannot pass through its membrane 18c, but molecule C can diffuse, see arrow, through membrane 18c from tubular well 16c into the culture in well 20. Therefore, with such an embodiment, three distinct biological moieties can be seeded in the individual wells of the top plate. While the three distinct biological moieties remain physically isolated from each other due to being contained in their own separate wells closed by semi-permeable membrane, the three distinct biological moieties can exchange molecules, such as the three molecules A, B, C, through the semi-permeable membrane into the community well in the bottom plate.

The apparatus can be used to culture many different combinations of biological moieties. For example, a plurality of different species of organisms can be cultured in one combination of insert wells and receiver well, as shown in FIG. 4A. Virus can be added to that system. Alternatively, a cell culture, such as of one or more mammalian cell, plant cell, immune cell, engineered cell types, may be cultured in the apparatus, in the receiver wells and/or in the insert wells. There may be more than one cell-type in the apparatus, combined together or separated in separate wells. There may also be combinations of a cell culture and one or more microorganisms and/or viruses. In one embodiment, the cell culture may be attached to the apparatus. In another embodiment, a substrate may be added to the insert well or the receiver well on which the cell culture is attached.

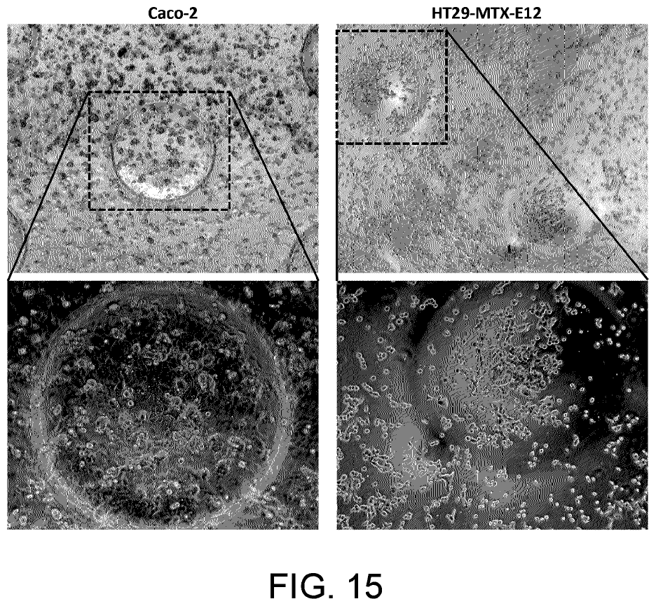

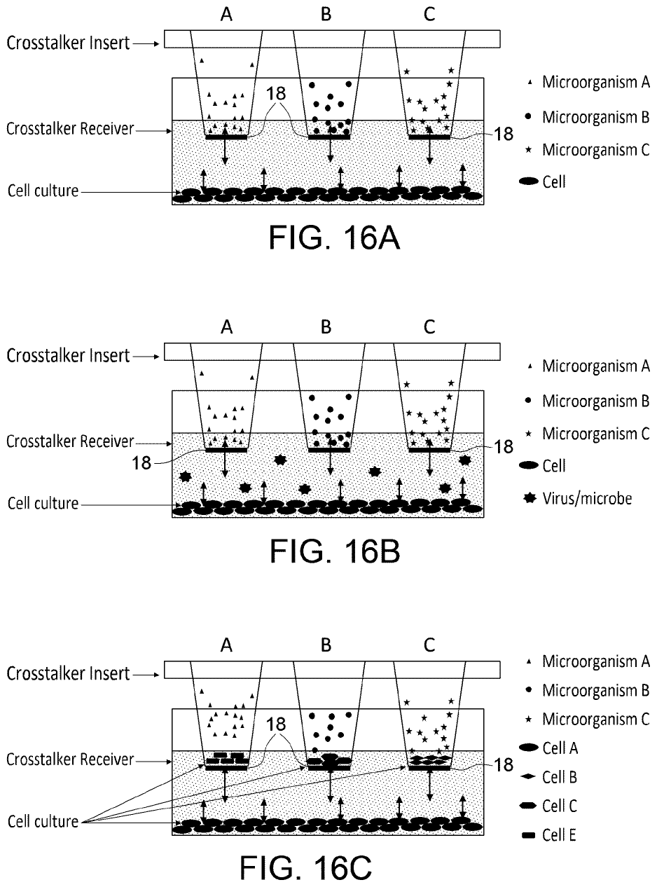

For example, as shown in FIG. 16A, there may be a cell culture including one or more cell type selected from a mammalian cell, a plant cell, an immune cell or an engineered cell, growing in the receiver well and there may be one or more microorganism, such as the three shown, cultured in the insert wells. While the cells of the cell culture can communicate via molecular diffusion through the membranes 18, the microorganisms are each unable to move out of their wells due to the exclusion action of the membranes. In this embodiment, the cell culture is immobile, supported on the bottom of the receiver well. However, the membrane can be selected to exclude any passage of the cells of the cell culture. In other words, the cell size or condition of the cells of the cell culture can are unable to pass through the membranes.

With reference to FIG. 16B, a virus or another microorganism can be added to one of the wells, such as to the receiver well, as shown. The membranes over the ends of the insert wells can be selected such that neither the microorganisms nor the additional virus/microbe can pass through. As such, the additional virus/microbe is trapped in the receiver well with the cell culture and the three microorganisms are each trapped in their insert wells, while molecular communication (see arrows) can occur between all the cells via diffusion through the membranes 18.

With reference to FIG. 16C, the apparatus can be employed to study a number of different cell cultures, such as cell cultures containing cell types A, B, C and D. In this embodiment, each one of the cell cultures are contained in their own well, selected from the receiver well and each of the three insert wells. In this embodiment, the membranes are each selected to exclude any passage of the cell culture cells. In other words, the cell size or condition of the cells of the cell culture can are unable to pass through the membranes. Thus, each cell culture can grow in its own well, but cannot move out of that well into other wells. The cell culture can grow on the membrane, but cannot pass through the membrane.

There are a range of semi-permeable membrane filter sizes from 10 nm to 20 μm pore size. The membrane pore size can be selected based on the application, for example, what is intended to be excluded from passage through the membrane. For example, in a bacterial application, where it is desired to culture a bacterial species in one well and prevent its diffusion into other wells of the apparatus, the size of the bacteria is considered and a pore size below that is selected to exclude (i.e. stop the passing) of the organism via the membrane. Generally, bacteria have a size above 0.2 μm and a membrane size of at least 0.2 μm is useful so that the microbe cannot pass through, but its metabolites, which have a size less than 0.2 μm, can pass through the membrane. In another example, virus particles are often sized from 80-130 nm. In an apparatus where the membrane is intended to stop the passage of virus from well to well, the membrane may have a pore size of 30-75 nm, for example 40-60 nm, to ensure that the virus cannot pass the membrane, while metabolites can diffuse through. A polymeric membrane, such as a polycarbonate membrane, is generally useful.

The apparatus can have all the insert well membranes the same pore size or different sizes. An apparatus with a number of different type membranes, such as with different pore sizes, can be used in one embodiment to study communication of different types of biological moieties with different sizes such as bacteria, virus or cell lines, simultaneously without mixing them in the community, such as shown in FIGS. 16A to 16C.

Both the upper and lower compartments (insert and receiver, respectively) can be easily accessed, allowing microbial cells or culture medium to be sampled, replaced, and analyzed from either compartment.

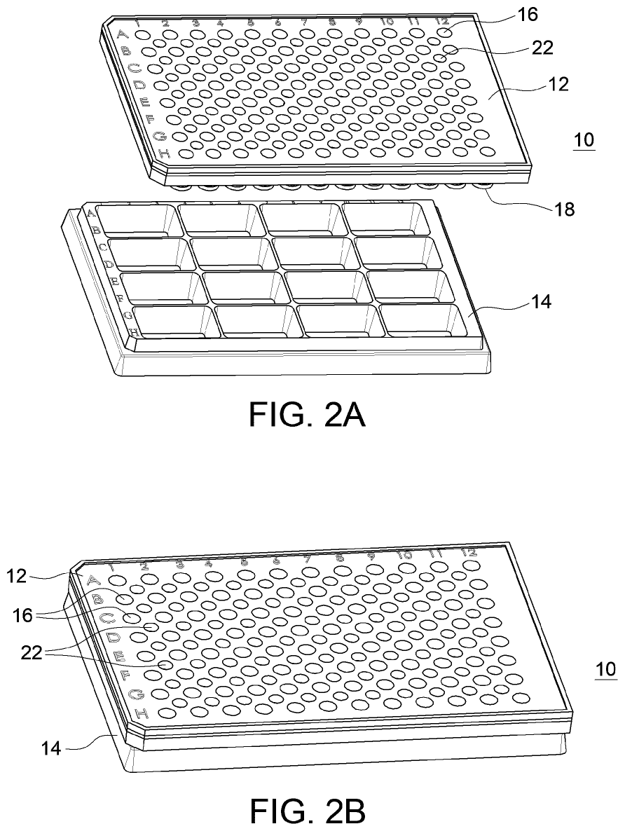

Because each tube is open on its upper end, there is access from above the insert plate down into the tube to its membrane extending thereacross to obtain samples of the culture medium in the tube. Samples of the culture medium in a well 20 can be obtained by lifting the insert plate 12 off the retainer plate. Alternatively, as shown in FIGS. 2A and 2B, insert plate 12 may include one or more access ports 22. Each access port is a hole passing fully through the insert plate and which is aligned over a well 20 in a retainer plate. The access ports may be spaced apart and distributed evenly across the insert plate. There may be more than one port 22 aligned with each receiver well, as it facilitates manufacture if each insert plate can fit over more than one receiver plate style. For example, the illustrated plate with 77 access ports can be used over a receiver plate with anywhere from 1 (i.e. receiver plate 20a FIG. 3) to 48 receiver wells (i.e. receiver plate 20b, FIG. 3) and even up to 77 receiver wells and provides access to each receiver well without removing the insert plate.

There is no permanent occlusion, such as a membrane, in the access port. Thus, the volume of the well 20 below the access port 22 can be accessed, without removing the insert plate. The access port may be sized for accepting passage therethrough of a pipette or syringe.

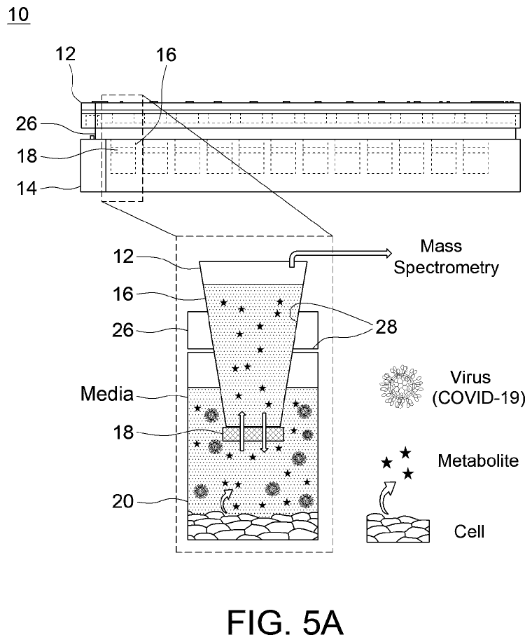

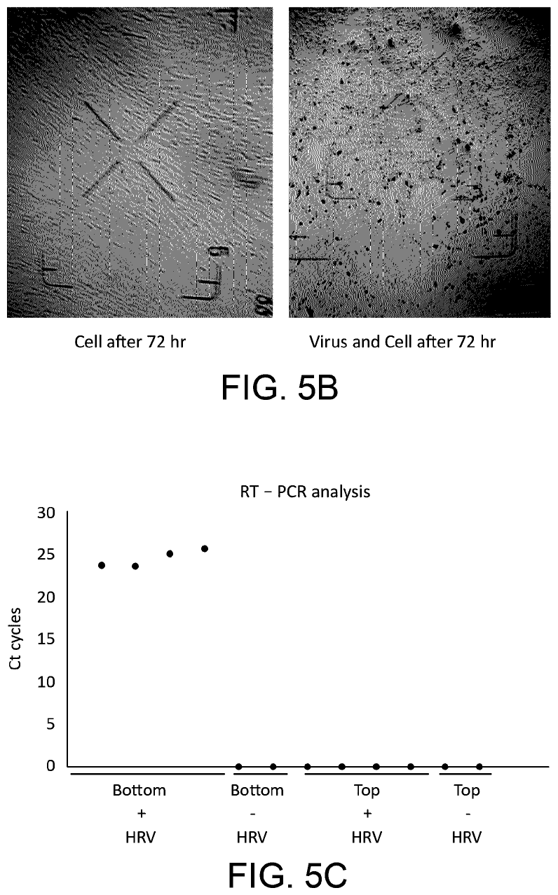

Some biological moieties, such as some viruses, may be hazardous for personnel. The present apparatus is useful for safe viral study, as it allows cells, such as microbes or virus-permissive cell lines to be grown using standard culture techniques. While the virus-containing culture may be hazardous, for example, it can be contained in the receiver plate. Therefore, the virus containing culture may be protected within the device, while access to uninfected culture medium can be had through the insert wells. The membrane can be selected to prevent passage from the receiver well up into the insert wells, while the virus-containing culture can still have access to oxygen and be exposed to treatments. In one embodiment with reference to FIG. 5, the apparatus 10 includes a gasket 26 and/or an adhesive 28 between the insert plate 12 and the receiver plate 14.

Gasket 26 seals between insert plate 12 and the lower, receiver plate 14 to create a leak-tight seal between the plates. Thus, the liquid cannot leak out of the receiver plate between the plates. Gasket 26 can be coupled to either or both of the insert plate 12 and the lower, receiver plate 14 and therefore in place, when the parts are fit together. Alternatively, the gasket can be provided separately from the plates and fit into place before the parts are fit together.

Alternatively or in addition, there may be adhesive 28 to hold the plates 12, 14 together. This adhesive prevents access to the receiver plate, so that personnel do not inadvertently access a hazardous material in the receiver plate. Also, the adhesives keeps the plates from falling apart and spilling the receiver plate contents inadvertently. In one embodiment, adhesive 28 is a permanent adhesive, so that once the insert plate is placed over the receiver plate with adhesive 28 between them and the adhesive sets, the insert plate is permanently secured and sealed to the lower plate. In one embodiment, the adhesive is, for example, applied to contacting surfaces between the plates, possibly protected with a peel sheet. Alternately, the adhesive can be provided as a separate sheet with a peel sheet on each side. The adhesive can be selected to create a permanent, non-reversible and leak-tight coupling between the plates.

The gasket and adhesive can be employed together. For example, gasket 26 can be permanently coupled to one of the plates with adhesive on its exposed side or the gasket can be provided separately from the plates with adhesive on both its upper and its lower sides, which are the sides that will contact the insert plate and the receiver plate, respectively. The adhesive can take many forms and configurations. In one embodiment, the adhesive is, for example, applied to the surface(s) of the gasket and protected with a peel sheet. Once the gasket is adhesively secured between the insert plate and the lower, receiver plate, the device cannot be separated and a leak-tight interface is formed at the interface between the insert plate and lower plate. Once the adhesive sets, which may be instantaneous, the device cannot be lifted or peeled from the lower plate and the only access to the culture is through the tubes of the insert plate and their membranes. However, the membranes can be selected to only permit passage of non-biohazard components of the receiver plate culture.

The gasket and/or adhesive can be configured to act around the edges of the insert plate and fit against the perimeter edges of the receiver plate, so there is no leak possible from the assembled apparatus. However, to maintain the culture in each receiver well, the gasket/adhesive may be positioned to encircle the upper rim of each well and, thereby, to create a seal at the interface between the insert plate and the upper rim of each receiver well. This maintains the culture, including a live biohazard, in each receiver well and prevents leaks from well to well. As noted, the apparatus can have many shapes and work with different plates configurations. However, just as an example, in one embodiment a 16-well receiver plate, the gasket can be configured to extend around the entire perimeter edge of the receiver plate and about each of the 16 wells. Thus, the gasket is in a sheet form with a rectangular outer edge and within the outer edge a thin grid of gasket sheet defining 16 holes, the grid being positionable on the upper rims of the wells.

The semi-permeable membrane allows researchers to conduct high throughput, micro-scale, reliable analyses. Apart from metabolite analysis via manual sampling, the apparatus allows microscopic and/or optical analyses as cells can be directly accessed through the various wells of the device.

A method for researching biological communities in culture can include: adding culture medium to an apparatus including a first well, a second well and a third well, wherein the first well and the second well are in fluid communication through a first semipermeable membrane and wherein the first well and the third well are in fluid communication through a second semipermeable membrane. Adding culture medium to the apparatus adds culture medium to the first well to at least make contact with the first and the second semipermeable membranes and wherein the culture medium contains a first molecule that can pass through the first semipermeable membrane.

The method further includes seeding a first biological moiety into the second well, the first biological moiety having a size that is unable to pass through the first semipermeable membrane; and seeding a second biological moiety into the third well, the second biological moiety having a size that is unable to pass through the second semipermeable membrane.

Thereafter, culture medium can be sampled from at least the second and third wells and possibly from any of the wells including the first, second and third wells.

In this method, the first biological moiety and the second biological moiety can be the same or different. These biological moieties can be selected from viruses or cells, such as prokaryotic cells (archaea, bacteria, etc.), yeast, mammalian cells, plant cells or immune cells. In one embodiment, the first biological moiety can be a first microbe and the second biological moiety can be a second microbe or the first biological moiety is a virus and the second biological moiety is a microbe or a cell, such as a human cell.

If desired, the second and third wells can be sealed and/or adhesively secured to the first well to prevent inadvertent access to the contents of the first well. In such an embodiment, a culture is created in the first well, for example, the culture media is added to the first well and the media is seeded with any of the selected virus, microbe, target cells, such as human cells, etc. Then a gasket and adhesive are used to permanently seal the second and third wells to the first well. This seals the culture in the first well. Once the adhesive sets, which may be instantaneous, the second and third wells cannot be lifted or peeled from the first well and the only access to the culture in the first well is through the second and third wells and their membranes. However, the membranes are selected to only permit passage of non-biohazard components of the culture out of the first well. The upper plate wells, which is the interior of the insert tube down to the membrane, can be easily and safely accessed, allowing culture medium to be sampled, replaced, and analyzed by various means without exposing laboratory staff to any live virus.

With reference to FIGS. 4B and 4C, for example, the method may include determining a metabolic contribution of individual biological moieties to an ensemble phenotype wherein the concentration of a metabolite is measured independently in both the first well and at least one of the second well and third well. In other words, biological moieties demonstrate different patterns of nutrient consumption and production when they are in a community with other biological moieties compared to when they are cultured in isolation. In a community, biological moieties respond to external molecular stimuli including signaling molecules or toxins produced by other biological moieties in the community. In an isolated in vitro system, the metabolic and molecular contributions from a biological moiety to a community comprised of multiple biological moieties cannot be measured. The apparatus enables the simultaneous measurement of molecules in several physically separated wells in a compartment, which enables the researcher to distinguish metabolic phenotypes or behaviors associated with both individual metabolism (when there is only one type of biological moiety present in adjacent wells of a compartment) and community metabolism (when there are multiple types of biological moieties present in adjacent wells of a compartment).

FIG. 4B is a diagram showing a calculation for a change in molecule concentration, herein molecule A, between an individual well and a community well. As shown,

Δ_{mA}=[Molecule A] _{individual in well 16}a−[Molecule A] _{community in well 20}.

This means that the net change in concentration of a given molecule produced or consumed by a biological moiety housed in an individual well can be calculated by subtracting the concentration of the molecule measured in the community well, where molecules are exchanged between adjacent wells, from the concentration of the molecule in the individual well housing the biological moiety. A positive net change in concentration indicates the molecule is produced by the biological moiety and a net negative change in concentration indicates the molecule is consumed by the biological moiety.

FIG. 4C is a diagram showing a calculation for a net contribution of a biology moiety to a collective molecule pool, wherein there are multiple biological moieties present and multiple molecules exchanged in the community well.

The net contribution of a biological moiety to the collective pool in well 20 is,
Δₘ, where m is a measured molecule in the system.

For each measured molecule in the system, Δₘ indicates whether that molecule was produced or consumed by an individual biological moiety, wherein a positive value indicates that the molecule was produced or excreted, and a negative value indicates that the molecule was consumed or taken in by the biological moiety.

In such an embodiment, a difference in a concentration of the metabolite between the selected well and the first well is used to calculate differential rates of metabolic production/consumption between the first biological moiety and the second biological moiety.

The sum of each Δₘ measured in the system by each biological moiety present in the system can be visualized to help the researcher understand the metabolic significance of the exchange of molecules in a community comprised of multiple biological moieties.

Alternatively or in addition, the method may be useful for assessing the growth of individual cells types in a community, wherein the growth of individual species within the community are independently monitored by measuring cell density. In such an embodiment, the method may include measuring cell density and may include calculating a change in cell density. This method may be measured by spectrophotometry (optical density at 600 nm) or colony counts.

The method may further comprise a quorum sensing (QS) wherein a small molecule modulates growth or other non-metabolic phenotypes of other cells in the community. Quorum sensing is a bacterial communication mechanism that controls gene expression based on the density of the bacterial population. Bacteria involved in quorum sensing produce and release chemical signals called autoinducers, whose levels increase as the cell population grows. The concentration of these autoinducers acts as an indicator of cell density. Quorum sensing that enables the regulation of various processes including biofilm formation, virulence factor expression, secondary metabolite production, and stress adaptation. This mechanism also encompasses bacterial competition systems, including secretion systems, which aid in the coordination of these activities.

### Summary of Uses

I. The apparatus enables biochemical and optical analyses of microbial communities and cell-cell interactions for any organism, including but not limited to proteins, carbohydrates, lipids, inorganics (i.e. salts), which may include signaling molecules and/or metabolites. Cells or organisms which may be analyzed in this manner include but are not limited to:
   a. Prokaryotic cells (archaea, bacteria)
   b. Yeast
   c. Mammalian cells
   d. Immune cells
   e. Plant cells
   f. Viruses.
II. The apparatus can be used to study drug interactions in a polymicrobial community, including but not limited to antibiotic susceptibility testing.
III. The apparatus enables metabolic flux analysis in a community of biological moieties, for example with a stable isotope labeled substrate.
IV. The apparatus can be used to study biofilm formation or quorum sensing in polymicrobial communities.
V. The apparatus can be used to study microbiome or model microbiome-related diseases and disorders.
VI. The apparatus can be used to evaluate environmental changes, for example light, pH, or nutrient levels, in a polymicrobial community or community with a host and other biological moiety.
VII. The apparatus can be used to study symbiotic, antagonistic, and neutral interactions between organisms in a community.
VIII. The apparatus can be used to model polymicrobial or viral infections.
IX. The apparatus can be used in a biosafety level 2 cabinet and does not require any microfluidics system for the assay or sampling.

The following examples are included for the purposes of illustration only, and are not intended to limit the scope of the invention or claims.

### Example I: Numerical Simulation

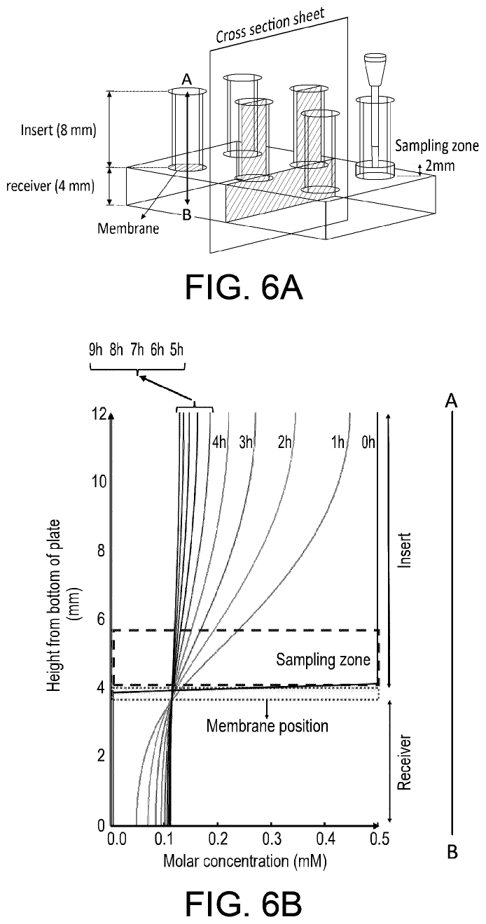

COMSOL Multiphysics™ simulation software, available from Comsol, Inc., was used to simulate the diffusion rate in an apparatus design, as shown in FIG. 6A. This study used initial concentrations of 0.00 mM and 0.50 mM on the bottom and top wells, respectively. The transport of diluted species physics and a time-dependent study was run over 9 hours to monitor the concentration change on the top and bottom wells. In addition, a mesh dependency test was run to ensure that the size and number of mesh nodes had no impact on the result. The results are shown in FIG. 6B. Mesh node refers to a specific point or location within a computational mesh. A computational mesh is a discretized representation of a physical domain used in numerical methods to solve equations or simulate phenomena.

The mesh is typically composed of interconnected elements, such as triangles or quadrilaterals in 2D or tetrahedra or hexahedra in 3D. These elements are defined by their vertices or nodes. Each node in the mesh represents a specific location in space and contains relevant information for the simulation, such as the values of variables being solved for (e.g., temperature, pressure) or other properties.

The mesh nodes serve as the fundamental building blocks of the numerical simulation, allowing the equations to be solved at specific points within the domain. By connecting the nodes with the elements, the entire computational domain is discretized into a mesh, enabling the numerical approximation of the desired solution.

### Example 2: Microbial Growth Assays

Microbial growth assays were prepared using Bacto™ Brain Heart Infusion broth (BHI; VWR International, LLC, Edmonton, AB, Canada, hereinafter VWR), prepared as per manufacturer's instructions and supplemented with Thayer Martin Supplement II (Sigma Aldrich, Oakville, ON, Canada), reconstituted as per manufacturer guidelines. The BHI broth was autoclaved at 121.5 °C for 20 min (Primus Sterilizer, Omaha, NE, USA) and cooled to room temperature before addition of the Thayer Martin supplement. 10 mL Thayer Martin Supplement II was added to 500 mL BHI medium and the resulting mixture was filter-sterilized (VWR Bottle-Top Filtration Unit, 500 mL, 0.1 um polystyrene membrane). The prepared medium was stored at 4 °C. Chocolate agar plates for overnight microbial growth were prepared as per manufacturer's instructions with BD Difco™ GC Medium Base (VR) and a 2% hemoglobin solution (BBL™ Freeze-Dried Hemoglobin Bovine Culture Media, VWR) supplemented with Thayer Martin Supplement II (Sigma Aldrich). All chemical standard solutions were prepared from compounds ordered from Sigma-Aldrich or Acros Organics (now part of Thermo Fisher Scientific, Waltham, MA, USA).

Staphylococcus aureus Rosenbach (ATCC® 25923™) and Pseudomonas aeruginosa (Schroeter) Migula (ATCC® 27853™) strains were purchased from the American Type Culture Collection (ATCC; Manassas, VA, USA). The Haemophilus influenzae strain was a clinical isolate obtained from Alberta Precision Laboratories (Calgary, AB, Canada). All bacterial stocks were stored at −80 °C until use. Each growth assay was repeated, as described below, on three separate days. All bacteria were grown overnight (approximately 18 hours) on chocolate agar plates in a Heracell™ VIOS 250i CO₂ Incubator (Thermo Fisher) at conditions of 36.5 °C, 5% CO₂ and 17.5% O₂. All experiments were performed in a Thermo 1300 Series A2 biosafety cabinet. Initial bacterial stock solutions were diluted to a total volume of 1.2 mL in liquid BHI medium, with initial optical density (OD) readings taken using a Thermo NanoDrop™ OneC Spectrophotometer at an absorbance of 600 nm. The stock solutions for H. influenzae and P. aeruginosa/S. aureus were diluted to OD₆₀₀ values of 0.9 and 0.5, respectively, and then transferred 1 mL of each stock solution into a sterile 150 mL Erlenmeyer flask containing 20 mL BHI medium. These solutions were shaken on a benchtop incubator (Benchmark Incu-Shaker™ Mini, Sayreville, NJ, USA) at 150 rpm for 2.5 hours to allow the bacterial cells to equilibrate to the liquid medium before measuring the resulting OD₆₀₀ values and diluting each stock solution to reach an OD₆₀₀ value of 0.1 in 10 mL BHI (H. influenzae) or 5 mL BHI (P. aeruginosa/S. aureus). For the P. aeruginosa and S. aureus cultures, then two 10-fold dilutions (100×) were performed to a final OD₆₀₀ value of 0.001 for these microbes in 10 mL BHI medium (the starting OD₆₀₀ value for H. influenzae remained at 0.1).

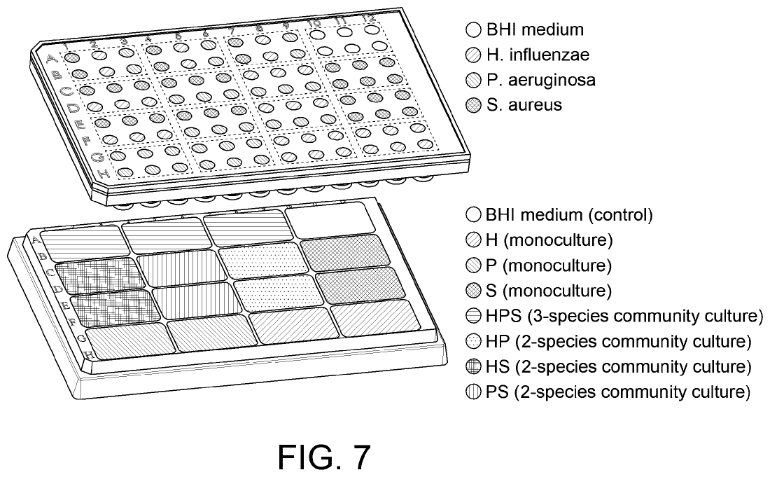

For the assay, an apparatus including a 96 well insert plate and 16 well receiver plate was used. Each insert plate well had a polycarbonate membrane sealed against its lower end. The membrane was 0.2 um pore size to prevent bacteria from moving into the receiver well. In this apparatus, six top wells fit into each receiver well. The apparatus was gas sterilized and placed in a biosafety cabinet. 100 μL of bacterial suspension with OD₆₀₀ value 0.1 for H. influenzae, 100 μL of bacterial suspension with OD₆₀₀ value 1×10⁻³ for P. aeruginosa, or 100 μL of bacterial suspension with OD₆₀₀ value 1×10⁻³ for S. aureus was transferred into separate individual top insert wells, according to the set up shown in FIG. 7.

Also, 2000 μL supplemented BHI medium (containing no microbes) was introduced to each receiver well. In this configuration, polymicrobial communities comprised of six individual wells each containing 100 μL bacterial suspension on the insert plate and one community well in the receiver plate were considered monocultures if all six individual wells contained the same microbe (i.e., H. influenzae, P. aeruginosa, or S. aureus). Other configurations including two or three of the microbial species mentioned were considered community cultures. In this setup, there was also one compartment that included six individual wells on the insert plate containing only the BHI medium (no microbial species) to serve as a control sample.

This setup allowed the observation of metabolic phenotypes associated with individual microbes in the monoculture compartments and phenotypes associated with polymicrobial communities in the community culture compartment, as well as comparison to the growth medium with no microbes present.

The apparatus was assembled with the insert plate installed on the receiver plate, with the membrane of the upper insert wells submerged in the receiver plate media. The apparatus was wrapped with a sterile gas-permeable film (rayon, 139.7 um pore size VWR) and the outside perimeter was wrapped with parafilm to avoid evaporation in the incubator. To avoid contamination and/or evaporation, a separate apparatus was set up for each metabolite sampling time point (9 hr, 18 hr) and growth density sampling timepoint (9 hr, 18 hr, 24 hr) and that apparatus was only removed from the incubator for sampling in the biosafety cabinet.

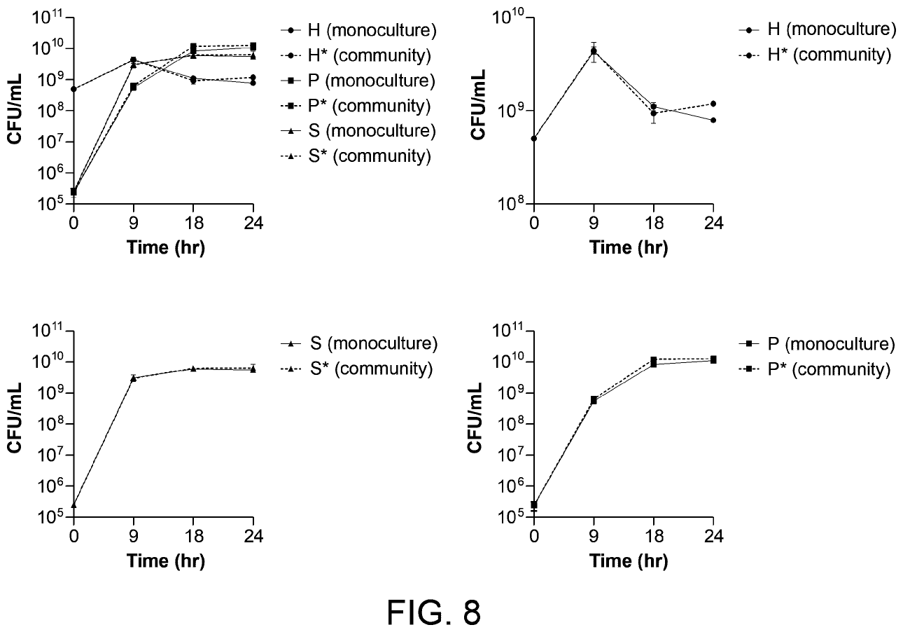

At 9 hr, 18 hr, and 24 hr (the latter for growth measurements only) the insert, top plate was removed from the receiver, bottom plate. The insert plate was set over a sterile 96-well plate (Falcon® 96-Well Cell Culture Plates, WWR) for sampling to avoid sample contamination or mixing. Samples were taken from the bottom, receiver wells, wherein 20 μL samples (3 replicates per well) were taken and each was diluted with 80 μL diluent (50% methanol (Fisher Optima)/50% water (Fisher Optima LC/MS grade)) in a 96-well plate (VWR 96-well Real-Time PCR skirted plate; 1:5 dilution). Before sampling from the top wells, each sample was mixed using a multi-channel pipet (at least 5 times per well) to release all bacterial cells that were adhered to the membranes. Next, 20 μL samples were extracted from each top well and diluted with 80 μL 50% methanol/water, as described for the bottom well sampling. Sampling plates were stored at −80 °C until all biological replicates were collected. 20 μL aliquots were extracted from separate but experimentally identical individual wells on the insert plate to perform optical density measurements and colony forming unit (CFU/mL) counts at each time point, ensuring enough volume was present in each sampled well for accurate measurements. FIG. 8 shows the CFU counts at the time periods 9, 18, and 24 hours. Colony forming units (CFU)/mL counts at each time point were taken by sampling a 20 μL aliquot from individual wells housing microbial species and diluting each aliquot by a factor of 10²-10⁶, followed by plating the diluted cells. Aliquots were taken from communities containing one microbial species (monoculture) and from communities containing three microbial species (community). H—Haemophilus influenzae; P—Pseudomonas aeruginosa; S—Staphylococcus aureus. FIG. 8 demonstrates that all microbial species were actively growing up to the 18 hour timepoint (the final metabolite sampling timepoint in this example). Six hours past this timepoint, each microbial species had colony counts equal to or greater than their initial colony counts. This indicates that the apparatus enables the growth of microbes over a longer period of time than in traditional in vitro growth experiments (usually performed for 8-12 hours) due to the large supply of growth medium contained in the receiver wells. Additionally, this indicates that all microbial species were in active metabolic states at both sampling timepoints (9 and 18 hours) so metabolic phenotypes observed are indicative of a metabolically active community in both monocultures and polymicrobial community cultures.

Optical density measurements were less consistent due to formation of biofilms and optically active compounds in later timepoints.

To prepare samples for metabolomics analysis, each sampling plate was centrifuged at 4,000 rpm for 5 minutes (Thermo Sorvall Legend XTR), after which 50 μL of the supernatant was transferred to a new 96-well sampling plate (Greiner 96 well plates, polypropylene, 392 μL/well; Monroe, NC, USA) and diluted with 150 μL of 50% methanol for a total sample dilution of 1:20 from the starting concentration. Samples were analyzed via ultra high-performance liquid chromatography-mass spectrometry (UHPLC-MS; Thermo Scientific).

Metabolite samples were resolved via a Thermo Fisher Scientific Vanquish UHPLC platform using hydrophilic interaction liquid chromatography (HILIC). Chromatographic separation was attained using a binary solvent mixture of 20 mM ammonium formate at pH 3.0 in LC-MS grade water (Solvent A) and 0.1% formic acid (% v/v) in LC-MS grade acetonitrile (Solvent B; Fisher Optima) in conjunction with a 100 mm×2.1 mm Syncronis™ HILIC LC column (Thermo Fisher Scientific) with a 2.1 μm particle size. A 15 minute gradient was used, as follows with percentages of Solvent B in the gradient shown: 0-2 min, 100% B; 2-7 min, 100-80% B; 7-10 min, 80-5% B; 10-12 min, 5% B; 12-13 min, 5-100% B; 13-15 min, 100% B. The flow rate used was 600 μL/min and the sample injection volume was 2 μL. Samples were ionized by electrospray using the following conditions: spray voltage of −2000 V, sheath gas of 35 (arbitrary units), auxiliary gas of 15 (arbitrary units), sweep gas of 2 (arbitrary units), the capillary temperature of 275 °C, the auxiliary gas temperature of 300 °C. Data were acquired on a Thermo Scientific Q Exactive™ HF Hybrid Quadrupole-Orbitrap™ Mass Spectrometer using full scan acquisitions (50-750 m/z) with a 240,000 resolving power, an automatic gain control target of 3e⁶, and a maximum injection time of 200 ms. All targeted data were acquired in negative electrospray ionization mode while the initial untargeted/semi-targeted data were acquired in both positive and negative electrospray ionization mode.

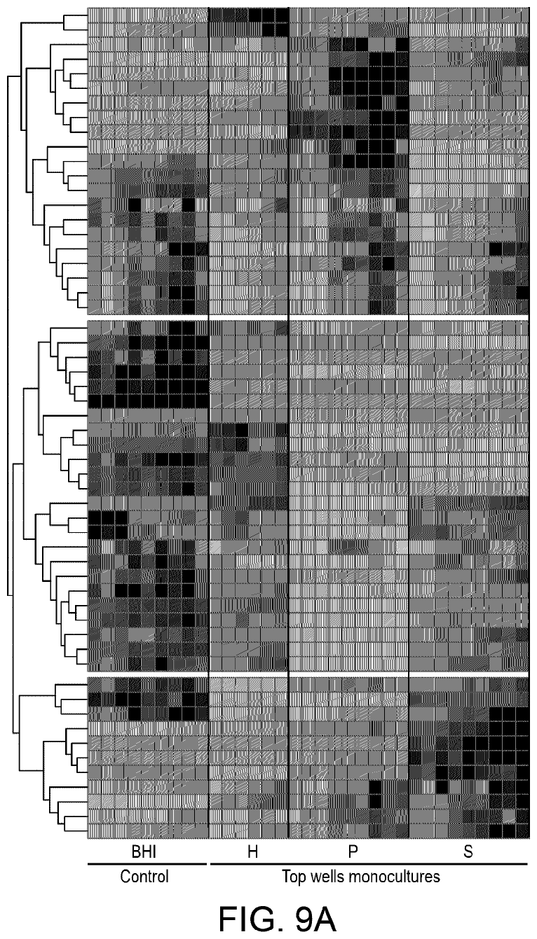

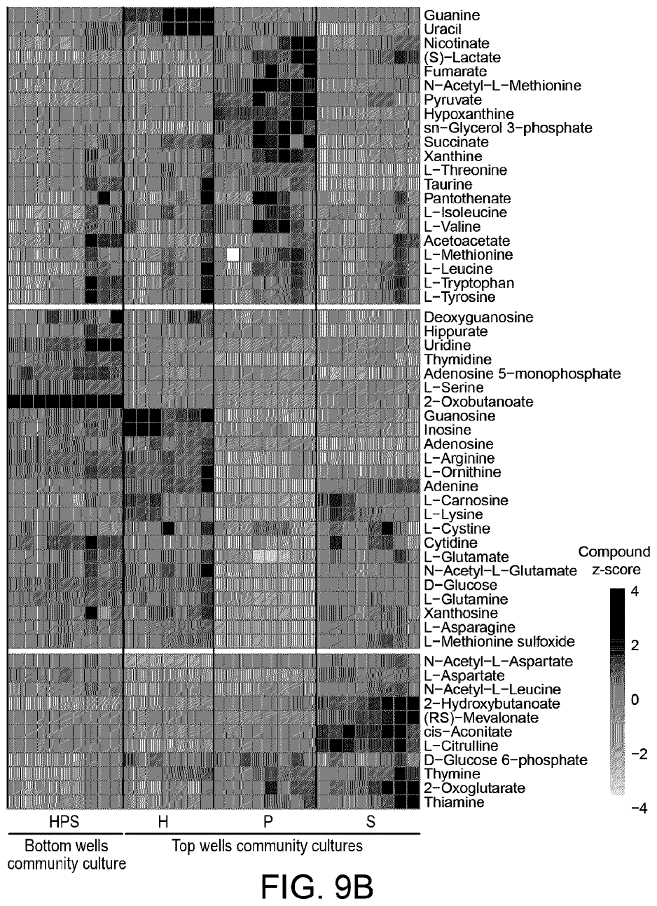

UHPLC-MS raw data were converted to mzXML file format using MSConvert GUI software and then conducted all MS analyses in El-Maven, version 12.0. Concentrations of each compound shown in the heatmap in FIGS. 9A and 9B were calculated by comparison to external standard curves. All statistical analysis and graphical visualizations of data were performed using GraphPad Prism™, version 9.4.0, where statistical differences between treatments were calculated by one-way ANOVA with post-hoc Tukey correction, where *p<0.05; **p<0.01; ***p<0.001; and ****p<0.0001. Other figures were generated using R Studio, version 4.2.3, and custom software developed in house.

FIGS. 9A and 9B is a heatmap showing initial screening of metabolites at the 9-hour time point, with metabolite values normalized by z-score across each row shown in the legend. The heatmap shows the row z-score for each metabolite, defined as the difference in metabolite concentration in an individual sample versus the mean concentration of that metabolite across all samples, divided by the standard deviation of the concentration of that metabolite across all samples. In other words, an individual sample showing a high z-score for a metabolite (in this example, shown in black) demonstrates a higher concentration for that metabolite compared to other samples. Conversely, an individual sample with a lower z-score for a metabolite (in this example, shown in light grey) demonstrates a lower concentration for that metabolite compared to other samples. The heatmap as a whole demonstrates patterns of metabolite consumption and production by different microbial species in a monoculture (one microbial species present in the compartment) versus their patterns of metabolite consumption and production in an ensemble phenotype of a community culture (three microbial species present in the compartment). For example, the metabolites in the top cluster of the heatmap are mainly produced by P. aeruginosa, as demonstrated by the larger number of dark-coloured boxes for P. aeruginosa compared to the other microbial species and the BHI control. The metabolites in the middle cluster of the heatmap are mainly produced by H. influenzae and the metabolites in the bottom cluster of the heatmap are mainly produced by S. aureus, of course with reference to the BHI control.

Sample legend: medium control—BHI; top well (insert plate) monocultures—H (Haemophilus influenzae), P (Pseudomonas aeruginosa), S (Staphylococcus aureus); bottom well (receiver plate) three-species culture-HPS; and top well (insert plate) three-species community cultures H* (H-HPS), P* (P-HPS), S* (S-HPS).

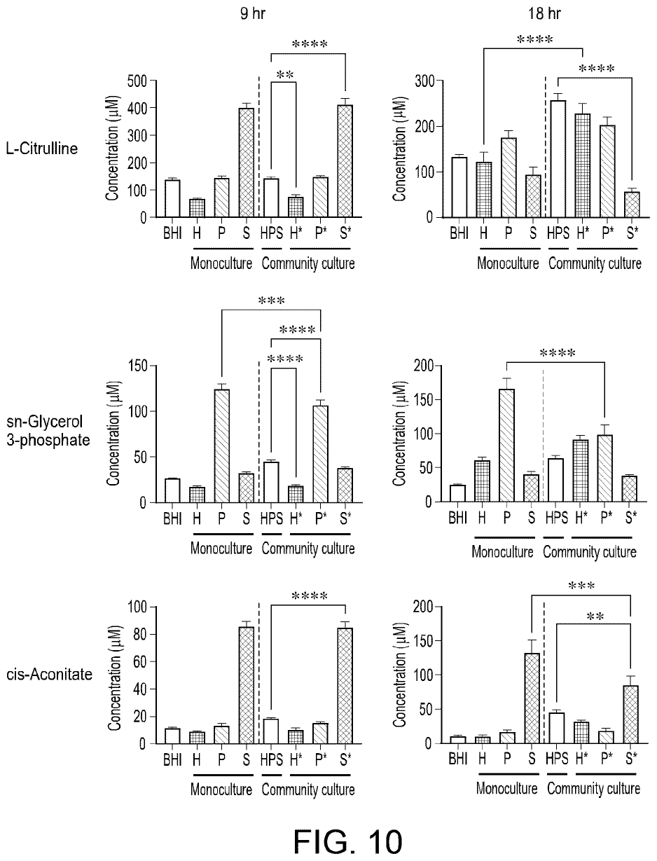

FIG. 10 is a series of bar plots showing metabolite concentrations of, from top to bottom, L-citrulline, sn-glycerol 3-phosphate and cis-aconitate, at 9 hours (left column) and 18 hours (right column). Error bars show the standard error of the mean. For each treatment, n=6-9 replicates and statistical differences between treatments were calculated by one-way ANOVA with post-hoc Tukey correction, where the asterisks denote significance levels when α=0.05 (*p<0.05; **p<0.01; ***p<0.001; and ****p<0.0001; only differences between the same microbe in different treatments or each microbe compared to the community well (HPS) are shown). These plots show that the apparatus can be used to distinguish significant changes in metabolite concentrations that occur due to a microbial species interacting with other microbial species in a community culture, compared metabolite concentrations of the microbial species in isolation (monoculture). Sample legend: medium control—BHI; top well (insert plate) monocultures—H (Haemophilus influenzae), P (Pseudomonas aeruginosa), S (Staphylococcus aureus); bottom well (receiver plate) three-species culture-HPS; and top well (insert plate) three-species community cultures H* (H-HPS), P* (P-HPS), S* (S-HPS).

Flux analysis of the monocultures, two-species, and three-species interactions were performed, according to the model described in FIG. 4B and equation below. Here D is the diffusion coefficient obtained from the data, which was experimentally determined to be 0.717 hr⁻¹. This was calculated by measuring the length of time for a molecule to pass through the semi-permeable membrane of the apparatus and, thus, reach equilibrium in both the insert plate and receiver plate.

Since this analysis is independent of how many compartments are connected to the bottom well, the equation of flux:

Flux=D(Cₜₒₚ−C_{bottom})

where D is the diffusion coefficient, Cₜₒₚ is the metabolite concentration in the top well of the insert plate, and C_{bottom} is the metabolite concentration in the bottom well in the receiver plate, can be used to determine fluxes of cells in different top wells connected to the same bottom well for two different time points.

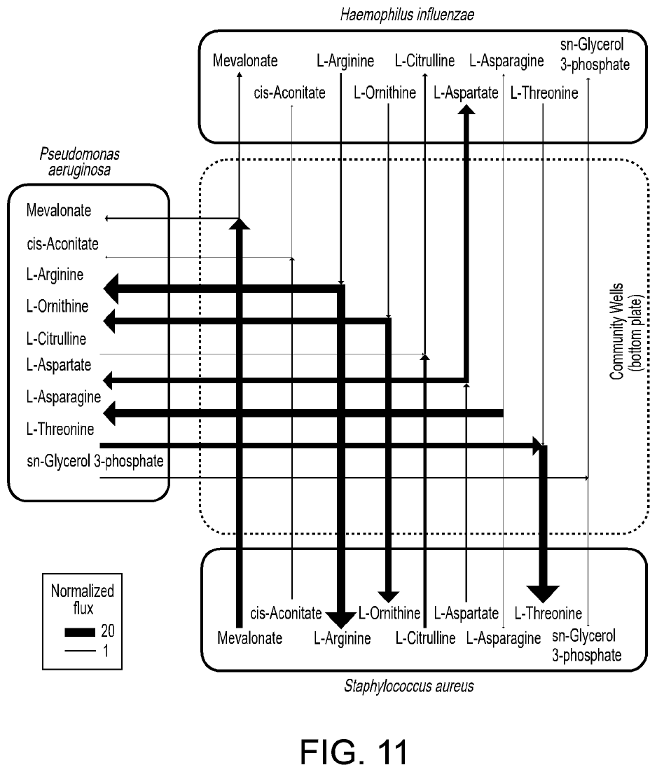

FIG. 11 illustrates metabolic flux observed between organisms at 9 hrs. In particular, the metabolic flux was calculated as described above between the three microbial species in a compartment containing three species housed in individual wells of the insert plate (H. influenzae, P. aeruginosa, and S. aureus) and a community well on the bottom (receiver) plate where the exchange of metabolites occurs. Flux values are normalized to the relative concentration of each metabolite and represented by the arrows, indicating both direction and magnitude of flux. This shows the overall metabolic contribution of individual microbial species to the community and visualizes the flow of metabolites from one microbial species to another microbial species in the community. This visualization differs from traditional in vitro systems with isolated microbial species, as it is not possible to determine the flow of molecules from one microbial species to another microbial species without a shared community well (in this case, the receiver plate).

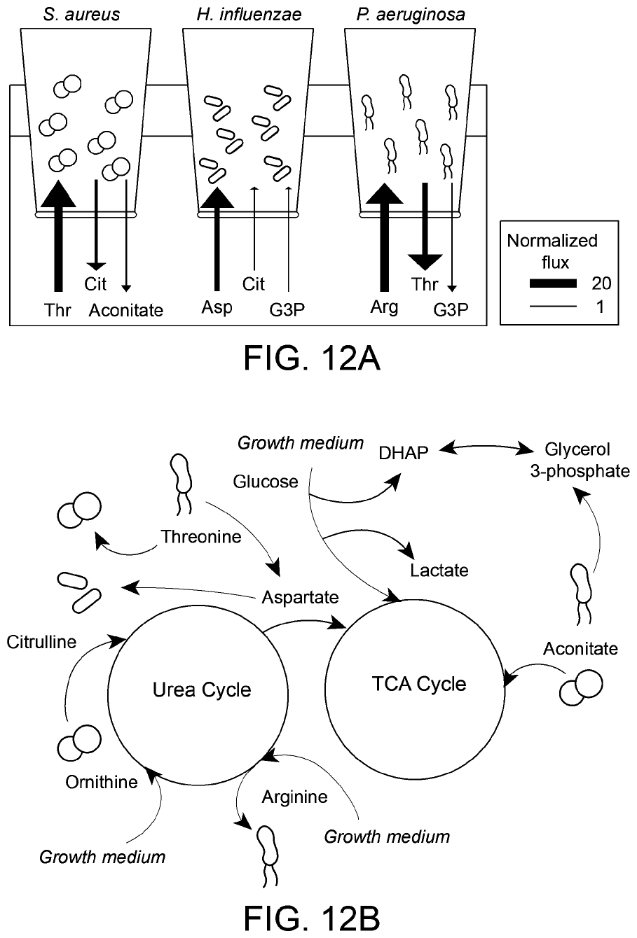

FIGS. 12A and 12B illustrate some examples of the biological relationships observed in Example 2 and shown in detail in FIG. 11. The data indicate that H. influenzae, with limited metabolic pathways compared to the other microbial species, consumes essential nutrients including amino acids (aspartate and citrulline) and energy sources (glycerol 3-phosphate) from the growth medium and the other microbial species. On the other hand, P. aeruginosa and S. aureus have more complete metabolic pathways and are able to take up nutrients from the growth medium and other microbial species and use their metabolic machinery to produce compounds that are then in turn consumed by other microbial community members. One example of this is the amino acid threonine, which is produced by P. aeruginosa and consumed by S. aureus, and can also be metabolically converted into the amino acid aspartate which is in turn taken up by H. influenzae. Visualizing the complex data resulting from the apparatus in the context of more complete metabolic pathways can elucidate the metabolic factors contributing to observed community metabolism phenotypes.

### Example 3—Cell Culturing and Viral Infection Procedure

MRC-5 (lung fibroblast) cells were grown and transferred into two 96-well plates after the cells reached 90% confluency and the Coronavirus 229E was incubated for 2 hours MOI 0.1, followed by replacing media with viral growth media (Eagle's minimum essential media TM (EMEM)+12% fetal bovine serum (FBS)) 150 ul in each well. A control plate was prepared with media and MRC-5 cells and a virus-infected plate was prepared with media and MRC-5 cells infected with virus (HRV).

The MRC-5 cell line was cultured in a receiver well on both plates and the receiver well of the virus-infected plate was also seeded with Coronavirus 229E, as shown in FIG. 5A. The membrane had 50 um pore size and was constructed of polycarbonate.

FIG. 5B shows a microscopic image of MRC-5 cell line cells from the control plate (Cell after 72 hr) and infected MRC-5 cells from the infected plate (Virus and Cell after 72 hr), each after 72 hrs.

Also, a sterility test was carried out by sampling media from the insert well. The media sample was assessed by polymerase chain reaction analysis and the result is presented in FIG. 5C. The PCR result of the sample taken from the insert well above the infected receiver well (Top+HRV), demonstrated that the virus in the receiver plate did not pass through the membrane and the insert plate remained sterile.

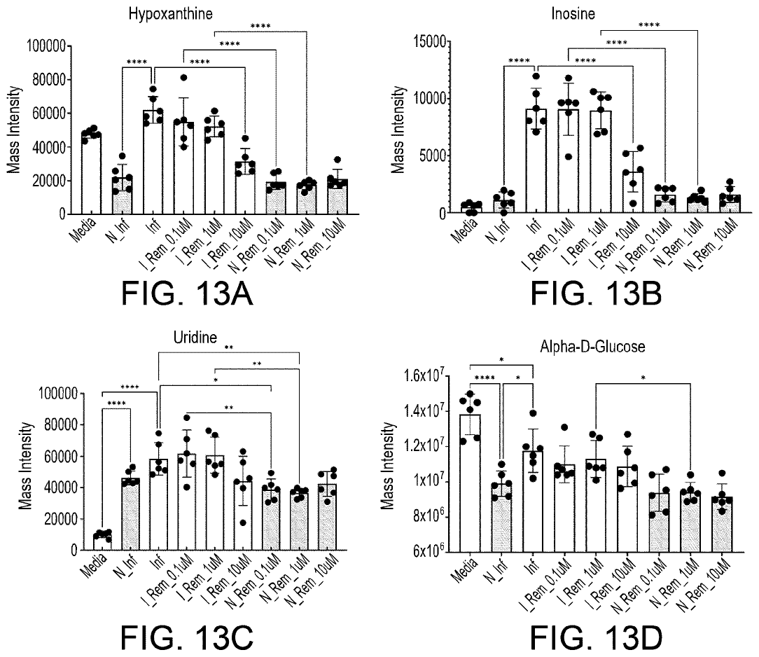

The apparatus of FIG. 5A was also utilized for a drug screening application. MRC-5 cells and Coronavirus 229E were used for testing Remdesivir (Rem) drug. Media samples were obtained from the insert wells of the apparatus and treated and analysed for metabolomics, as in Example 1. FIG. 13 shows metabolomics results for (a) Hypoxanthine, (b) Inosine, (c) Uridine and (d) Alpha-D-Glucose.

### Example 4—Biofilm Formation

An apparatus according to the invention was utilized for a biofilm formation study.

A Calgary Biofilm Device (CBD) was used to measure biofilm formation. A CBD is a lid-type device with cone shape pegs extending from its underside. The pegs are spaced apart and sized to fit down over a culture well with the pegs extending down into the culture well. The CBD is useful to characterize and measure biofilm formation.

In this example, a CBD with 96 cone shape pegs was selected to fit down over a 96 well insert plate according to the invention. Each peg extended down into one of the insert wells: one peg in one insert well.

The insert wells were each inoculated with bacteria and the pegs of the CBD inserted therein. Specifically, a community was set up with S. aureus (SA) in one insert well, H. Influenza (HI) in an adjacent insert well and P. aeruginosa (PA) in an adjacent insert well and all three of the insert wells inserted into the same receiver well with BHI medium. All materials and methods with respect to bacterial samples and innoculations according to Example 2. All insert wells had a 0.2 um pore size membrane sealed against the lower end. This community resembled the set up of FIG. 4A.

With reference to wells 16a and 16b of FIG. 4A, further two microbe communities of (a) S. aureus (SA) and H. Influenza (HI), (b) S. aureus (SA) and P. aeruginosa (PA) and (c) H. Influenza (HI) and P. aeruginosa (PA) were seeded.

Then, monocultures of S. aureus (SA), H. Influenza (HI) and P. aeruginosa (PA) and a control of only media were each prepared.

The CBD lid was installed over the apparatus, such that the pegs were submerged in the media. The assembled plate was cultured for 48 hrs.

Crystal violet staining was used to quantify the biofilm after 48 hrs of incubation. The pegged lids from the CBD were rinsed twice in 200 μl of 0.9% saline. The biofilms were stained with 200 μl of a 0.1% crystal violet solution for 30 min. Following staining, the pegs were washed with 200 μl ddH₂O three times to remove excess dye. Quantification of the biofilm was performed by sonication using a 250HT ultrasonic cleaner (VWR), set at 60 Hz for 10 min into 200 μl of 70% ethanol and reading the absorbance at 590-640 nm.

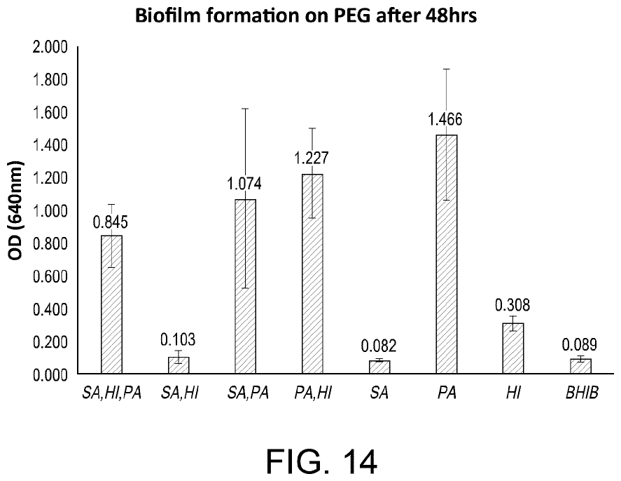

FIG. 14 shows the results for S. aureus (SA), H. Influenza (HI) and P. aeruginosa (PA) intractions. The result demonstrates that the monoculture P. aeruginosa (PA) had higher biofilm formation compare to other two and three bacteria communities. In another words, P. aeruginosa (PA) formed less biofilm in a community compared to its monoculture bacteria.

### Example 5—Mammalian Cell Culturing

To demostrate the utility of an apparatus according to FIG. 1 for culturing a mammalian cell culture, Caco2 cells (cell line of human colorectal adenocarcinoma cells) with EMEM (10% FBS, NA 1%, Penicillin-Streptomycin 1%) were inserted (400k/well) in to each well of a receiver plate and incubated for 24 hours in a cell culture incubator (37 °C). Also another cell line, HT29-MTX-E12 with Dulbecco's Modified Eagle Medium (DMEM) media (10% FBS, glutamax 1%, pen/strep 1%) was cultured (400k per crosstalker receiver well) and incubated for 24 hours. A Nikon TE 2000 microscope was utilized to visualize the cultured cells after 24 hours, as shown in FIG. 15. The results show that cells are fully attached and growing in their receiver wells.

The previous description and examples are to enable the person of skill to better understand the invention. The invention is not be limited by the description and examples but instead given a broad interpretation based on the claims to follow.

1. A laboratory apparatus for research of a community of biological moieties comprising:
a. a receiver plate with a receiver well;
b. an insert plate for positioning over the receiver plate, the insert plate including a first tubular well extending from the lower surface and a second tubular wall extending from the lower surface, each of the first and second tubular wells including an upper open end and a lower end that is open, the first and second tubular wells are configured such that the lower ends protrude into the receiver well when the insert plate is positioned over the receiver plate; and
c. a first membrane extending across the lower end of the first tubular well and a second membrane extending across the lower end of the second tubular well, the first membrane selected to exclude a first cultured biological moiety, while permitting molecules smaller than the first cultured biological moiety of a culture medium to pass through and the second membrane selected to exclude a second cultured biological moiety, while permitting molecules smaller than the second cultured biological moiety of a culture medium to pass through.

2. The laboratory apparatus of claim 1 wherein the first membrane has a pore size of up to 0.2 um.

3. The laboratory apparatus of claim 1 further comprising an access port passing through the insert plate adjacent to but spaced from the first tubular well and aligned over the receiver well.

4. The laboratory apparatus of claim 1 further comprising a permanent adhesive between the insert plate and the receiver plate.

5. A method for researching biological systems with a laboratory apparatus as in claim 1, comprising: seeding a first cell type into the first tubular well and seeding a biological moiety into at least one of the receiver well and the second tubular well; and determining a metabolic contribution of the first cell type and the biological moiety to an ensemble phenotype, wherein a concentration of a metabolite is measured independently in both the first tubular well and the at least one of the receiver well and the second tubular well.

6. The method of claim 5 wherein a difference in a concentration of the metabolite between the first tubular well and the at least one of the receiver well and the second tubular well is used to calculate differential rates of metabolic production/consumption between the first cell type and the biological moiety.

7. The method of claim 5 wherein the biological moiety is a virus and the virus is seeded into the receiver well.

8. A method for researching biological systems with a laboratory apparatus as in claim 1, comprising: seeding a first cell type into the first tubular well and seeding a biological moiety into at least one of the receiver well and the second tubular well; and assessing growth of the first cell type.

9. The method of claim 8, wherein assessing growth includes measuring cell density.

10. The method of claim 8, wherein assessing growth is by use of an optical density measurement or by colony counts.

11. The method of claim 8, wherein the biological moiety is a virus and the virus is seeded into the receiver well.

12. The method of claim 8, wherein the biological moiety is a second cell type, and the method further comprises assessing a community phenotype in culture medium in the receiver well based on metabolic contributions by both the first cell type and the second cell type.

13. The method of claim 5 wherein the first cell type is a mammalian cell, a plant cell, an immune cell, an engineered cell, a microorganism, a bacteria or a yeast.

14. A method for researching biological communities in culture, the method comprised of:
a. adding culture medium to a container system including a first well, a second well and the a third well, wherein the first well and the second well are in fluid communication through a first semipermeable membrane and wherein the first well and the third well are in fluid communication through a second semipermeable membrane, and adding culture medium to the container system adds culture medium to at least make contact with the first and the second semipermeable membranes and wherein the culture medium contains a molecule that can pass through the first and the second semipermeable membranes; and
b. seeding a first biological moiety into the second well, the first biological moiety having a size that is unable to pass through the first semipermeable membrane;
c. seeding a second biological moiety into the third well, the second biological moiety having a size that is unable to pass through the second semipermeable membrane; and
d. sampling culture medium from the second well.

15. The method of claim 14, wherein the first biological moiety is a first microbe and the second biological moiety is a second microbe.

16. The method of claim 14, wherein the first biological moiety is a virus and the second biological moiety is a microbe.

17. The method of claim 14, wherein the first biological moiety is a mammalian cell and further comprising seeding a virus into the first well.
